(19) United States
(12) Patent Application Publication
(10) Pub. No.: US 2023/0263469 A1
(43) Pub. Date: Aug. 24, 2023

(54) SHOE-BASED SENSOR SYSTEM FOR DETECTING HUMAN MOTION AND BODY BALANCE

(71) Applicant: The Board of Trustees of The University of Alabama, Tuscaloosa, AL (US)

(72) Inventors: Zachary Choffin, Batavia, IL (US); Nathan Seongheon Jeong, Tuscaloosa, AL (US); Michael Callihan, Northport, AL (US); Savannah Olmstead, Colorado Springs, CO (US); Edward Sazonov, Northport, AL (US)

(21) Appl. No.: 18/163,994

(22) Filed: Feb. 3, 2023

Related U.S. Application Data
(60) Provisional application No. 63/313,567, filed on Feb. 24, 2022.

(51) Int. Cl. A61B 5/00; A61B 5/107; A61B 5/103; A61B 5/0205; A61B 5/11

(52) U.S. Cl. CPC A61B 5/6807; A61B 5/1071; A61B 5/1036; A61B 5/02055; A61B 5/112; A61B 2562/0219

(57) ABSTRACT

A system for detecting human motion and body balance includes a flexible substrate configured to be positioned in a shoe of a user, a sensor array comprising one or more force sensors positioned on the flexible substrate, and a controller communicably coupled to the sensor array. The controller is configured to receive force data from the sensor array, determine biomechanical characteristics for the user based on the force data, the biomechanical characteristics including at least one of a center-of-pressure, a center-of-mass, or lower extremity angles for the user, and provide an indication of the biomechanical characteristics to the user.

### CROSS-REFERENCE TO RELATED APPLICATIONS

This application claims the benefit of and priority to of U.S. Provisional Patent App. No. 63/313,567, filed Feb. 24, 2022, which is incorporated herein by reference in its entirety.

### BACKGROUND

The present disclosure relates generally to a wearable motion and balance detection system and associated methods of determining body mechanics for a user.

In recent years, the market for sensors for wearable devices has grown at a rapid pace with the ever-increasing demand for wearable activity trackers, smartwatches, smartphones, and the like. Many activity trackers and smartwatches, for example, may include sensors for measuring a user's heartrate, tracking the user's steps, detecting when the user is performing an exercise, etc. However, many currently used sensor systems are not capable of providing real-time analysis and prediction of human motion and body balance or, at best, they provide inaccurate and erroneous motion and balance predictions. A smartwatch may make use of an inertial measurement unit (IMU) to detect a user's steps, for example, but most smartwatches are incapable of evaluating the user's body mechanics and providing meaningful feedback.

Some state-of-the-art motion capture systems exist for capturing and analyzing human motion in real-time; however, these systems are often complex, bulky, and too costly for general users. As an example, these motion capture systems can require any of a plurality of cameras, a computerized tomography (CT) scanner, an X-ray scanner, multiple IMUs, etc. Even motion capture systems that only use a plurality of cameras still often require a plurality of optical markers to be positioned at various points on a user. Thus, these types of motion capture systems are not feasible for daily use due to the complex set-up processed and mechanical instability of said systems when in motion.

### SUMMARY

One implementation of the present disclosure is a system for detecting human motion and body balance. The system includes a flexible substrate configured to be positioned in a shoe of a user, a sensor array including one or more force sensors positioned on the flexible substrate, and a controller communicably coupled to the sensor array. The controller is configured to receive force data from the sensor array, determine biomechanical characteristics for the user based on the force data, the biomechanical characteristics including at least one of a center-of-pressure (CoP), a center-of-mass (CoM), or lower extremity angles for the user, and provide an indication of the biomechanical characteristics to the user.

In some embodiments, providing the indication of the biomechanical characteristics to the user includes transmitting, by the controller, data including the biomechanical characteristics to a user device associated with the user to cause the user device to display a graphical user interface (GUI).

In some embodiments, the controller is further configured to detect an abnormality in the biomechanical characteristics for the user.

In some embodiments, the indication further includes a recommendation for correcting the abnormality.

In some embodiments, the lower extremity angles include a predicted angle for each of an ankle, a knee, a hip, and a lower back of the user.

In some embodiments, determining the biomechanical characteristics for the user includes calculating the lower extremity angles using a machine learning model.

In some embodiments, the force data is provided as an input to the machine learning model, the machine learning model configured to predict an angle for each of an ankle, a knee, a hip, and a lower back of the user.

In some embodiments, the one or more force sensors include force sensing resistors (FSRs), piezoresistive (PZR) sensors, capacitive force sensors, or fabric-based strain sensors.

In some embodiments, the flexible substrate is an insole of the shoe or wherein the flexible substrate is configured to be positioned under an insole of the shoe.

In some embodiments, the one or more force sensors include at least three sensors, where a first subset of the one or more force sensors is positioned on the flexible substrate to be under a heel of the user's foot; a first subset of the one or more force sensors is positioned on the flexible substrate to be under a ball of the user's foot; and a first subset of the one or more force sensors is positioned on the flexible substrate to be under a toe of the user's foot.

In some embodiments, the system further includes at least one inertial measurement unit (IMU) positioned on the flexible substrate.

In some embodiments, the controller is configured to receive at least one of angular rate data or specific force data from the at least one IMU and calculate the CoM for the user based further on the at least one of angular rate data or specific force data.

In some embodiments, the system further includes at least one additional sensor positioned on the flexible substrate for measuring one or more biometric characteristics of the user.

In some embodiments, the one or more biometric characteristics of the user include at least a heart rate and an oxygen saturation of the user.

In some embodiments, the at least one additional sensor is positioned on the flexible substrate to be under a lateral portion of the user's foot.

In some embodiments, the system further includes at least one of a temperature sensor or a moisture sensor positioned on the flexible substrate.

In some embodiments, the temperature sensor is configured to measure a temperature of an interior of the shoe and the moisture sensor is configured to measure a humidity level in the shoe.

In some embodiments, the moisture sensor is positioned on the flexible substrate to be under a toe of the user's foot.

In some embodiments, the system further includes an antenna positioned on the flexible substrate for detecting a gait pattern of the user, where the gait pattern of the user is detected by measuring a resonant frequency of the user's foot during a gait cycle using the antenna.

In some embodiments, the antenna is one of a coupled antenna, a meandered antenna, or a spiral antenna.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram of a sensor system for detecting human motion and body balance, according to some embodiments.

FIGS. 2A-2E are diagrams illustrating various configurations of an in-shoe sensor array for the shoe-based sensor system of FIG. 1, according to some embodiments.

FIGS. 3A and 3B illustrate an example implementation of the shoe-based sensor system of FIG. 1, according to some embodiments.

FIG. 4 is a flow diagram of a process for determining various body mechanics of a user using the shoe-based sensor system of FIG. 1, according to some embodiments.

FIG. 5 is an example diagram illustrating center-of-pressure (CoP) detection using the shoe-based sensor system of FIG. 1, according to some embodiments.

FIG. 6 is an example diagram illustrating center-of-mass (CoM) detection using the shoe-based sensor system of FIG. 1, according to some embodiments.

FIGS. 7A and 7B are example user interfaces for providing feedback regarding a user's body mechanics, according to some embodiments.

FIG. 8 is a flow diagram of a process for predicting the positioning (e.g., angle) of a user's lower extremities using the shoe-based sensor system of FIG. 1, according to some embodiments.

FIGS. 9A-9D are graphs showing the prediction of various lower extremity angles using the process of FIG. 8, according to some embodiments.

FIGS. 10A and 10B are example user interfaces for providing feedback regarding a user's biometric characteristics, according to some embodiments.

FIGS. 11A-11G are diagrams of various antenna configurations for detecting a user's gait, according to some embodiments.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and to the Figures and their previous and following description.

Overview

Referring generally to the figures, a system for detecting and tracking user movement and body mechanics is shown, accordingly to various embodiments. More specifically, the system described herein includes a sensor array that is positioned on a flexible substrate intended to be placed under, or to replace, the insole of a user's shoe. In other words, the sensor array is shoe-based (i.e., worn in the user's shoe(s)). The sensor array may include a plurality of force sensors that are configured to measure the force exerted by the user at various points of the user's foot. In this manner, various aspects of the user's motion (e.g., ground reaction forces) can be detected and tracked in real or near-real time, as will be discussed in greater detail below. In some configurations, additional devices such as inertial measurement units (IMUs), biometric sensors, environmental sensors, and antennas for detecting a user's gait may also be positioned on the flexible substrate for providing additional biometric and biomechanical feedback to the user.

In addition to the motion and balance detection system, various methods are described herein for analyzing the user's body mechanics. For example, data from the sensor array mentioned above can be utilized to predict the angles of a user's lower extremities (e.g., ankles, knees, hips, and lower back). This type of body mechanic (i.e., biomechanical) data can then be used to provide recommendations to the user to help them improve posture, gait, weight distribution, etc., which can help to prevent injuries and long-term health issues. Thus, the system and methods described herein can provide a robust analysis of a user's body mechanics and biometrics in a compact, comfortable, and affordable device. Additional features and advantages are described in greater detail below.

Shoe-Based Sensor System

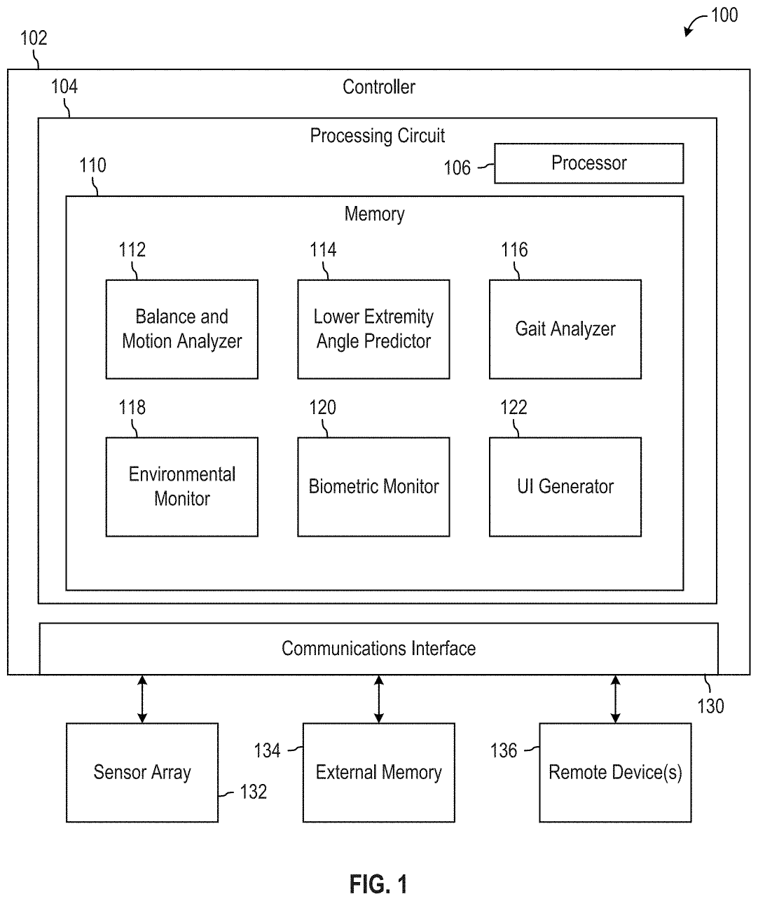

Turning first to FIG. 1, a block diagram of a shoe-based sensor system 100 for detecting human motion and body balance is shown, according to some embodiments. System 100, also referred to herein as motion and balance detection system 100, may be configured to detect and track various biomechanical and biometric characteristics of a user. For example, system 100 may be configured to measure an amount of force (i.e., pressure) exerted by the user against the ground as they walk (e.g., ground reaction force) and/or stand using a sensor array 132, which is described in greater detail below. Based on recorded force data, the user's body mechanics (e.g., gait, posture, weight distribution, etc.) can be calculated and/or predicted, which can not only give the user valuable insight into their physiological health but can also be used to provide (e.g., by system 100) recommendations for the user to improve their body mechanics.

System 100 is shown to include a controller 102 that is in communication with sensor array 132. As used herein, “communication” may refer to bidirectional electronic communication (e.g., the sending and receiving of data) between at least two components of system 100. Accordingly, a first component that is “communicably coupled” to a second component may, in general, transmit data to and receive data from the second component; although it will be appreciated that only “communicably coupled” may indicate that only one component is receiving/transmitting data. In any case, controller 102 may receive data from sensor array 132 and, in some embodiments, may transmit control signals and/or power to sensor array 132.

As shown, controller 102 may include a communications interface 130 to facilitate the transfer of signals (e.g., data, power, etc.) between controller 102 and a variety of external components, including sensor array 132. Accordingly, communications interface 130 can be and/or can include a wired and/or wireless communications interface (e.g., jacks, antennas, transmitters, receivers, transceivers, wire terminals, etc.) for conducting data communications. In various embodiments, communications via communications interface 130 may be direct (e.g., local wired or wireless communications) or via a network (e.g., a WAN, a LAN, a VPN, the Internet, a cellular network, etc.). For example, communications interface 130 can include a WiFi® transceiver for communicating via a wireless communications network. In another example, communications interface 130 may include cellular or mobile phone communications transceivers. In yet another example, communications interface 130 may include a low-power or short-range wireless transceiver (e.g., Bluetooth®). Thus, it will be appreciated that communications interface 130 may be configured to transmit and receive data by any suitable means and/or on any suitable try of network. In some embodiments, communications between controller 130 and any remote components (e.g., sensor array 132, an external memory 134, or remote devices 136), described below, may be encrypted for added security. For example, an end-to-end encryption (E2EE) protocol may be established such that any of controller 102, sensor array 132, an external memory 134, and remote devices 136 may transmit encrypted data than can only be decrypted by one of the other components.

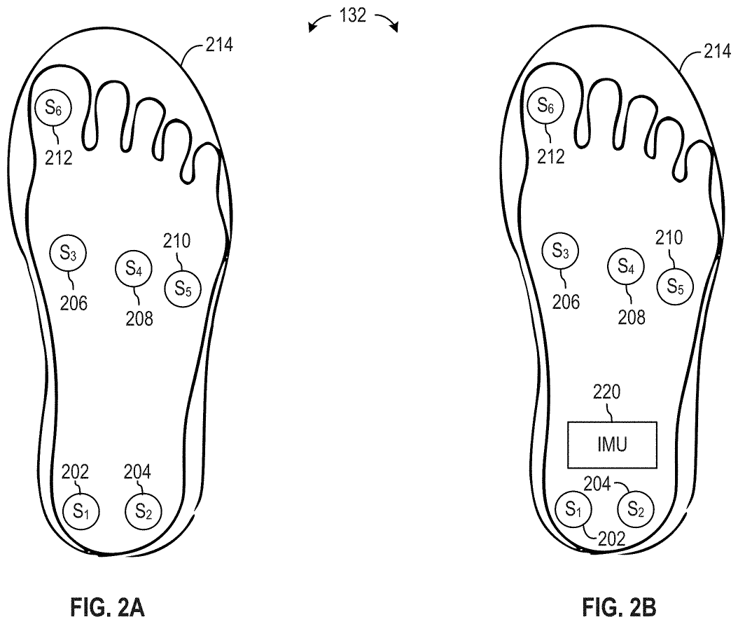

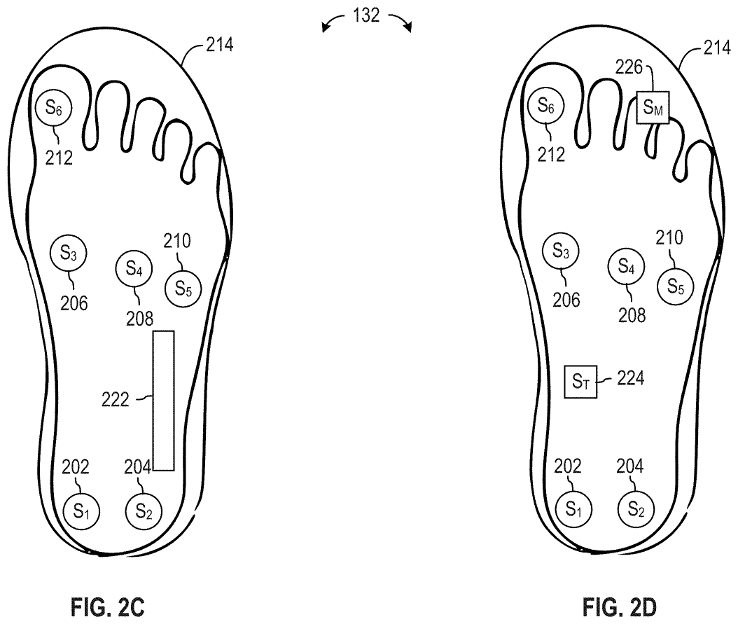

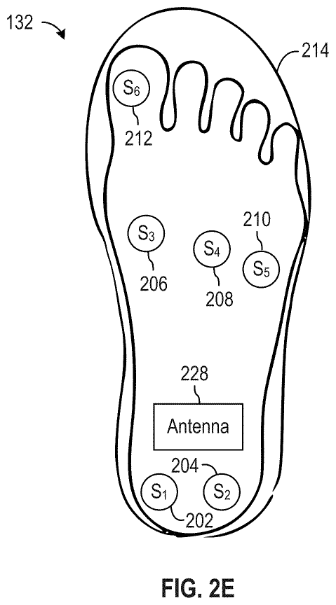

Sensor array 132, as briefly mentioned above, may include a variety of sensors for measuring various biomechanical and biometric characteristics associated with a user. In some embodiments, the variety of sensors included in sensor array 132 are positioned on, or embedded into, a flexible substrate, as described in greater detail below with respect to FIGS. 2A-2E. In some such embodiments, the flexible substrate is configured to be placed under an insole of the user's shoe(s). In other such embodiments, the flexible substrate is configured to replace the insole of the user's shoe(s). Accordingly, sensor array 132, and by extension system 100, may be much more compact and comfortable than other types of systems for measuring human motion, body balance, and other biomechanical characteristics. In this regard, system 100 may be much more practical for daily and/or casual use by a wide variety of users.

In some embodiments, sensor array 132 includes a plurality of force (i.e., pressure) sensors configured to measure an amount of force (i.e., pressure) exerted by the user against the ground as they walk (e.g., ground reaction force) and/or stand. In some embodiments, sensor array 132 further includes one or more IMUs for measuring angular rate and specific force as the user moves, which can be used to determine a position and distance between the user's shoes. In some embodiments, sensor array 132 includes an antenna for measuring and recording the user's gait. In some embodiments, sensor array 132 also includes sensors for measuring various biometric characteristics of the user, such as heartrate and oxygen (O₂) saturation. In some embodiments, sensor array 132 also includes sensors for measuring various environmental characteristics (e.g., in the user's shoe). For example, sensor array 132 may include one or more temperature sensors for measuring a temperature in the user's shoe and/or may include one or more moisture sensors for measuring a humidity or moisture level in the user's shoe.

As shown in FIG. 1, in some embodiments, controller 102 can communicate with external memory 134. External memory 134 can include one or more devices (e.g., memory units, memory devices, storage devices, etc.) for storing data and/or computer code for completing and/or facilitating the various processes described in the present disclosure. In some embodiments, external memory 134 includes tangible, computer-readable media that stores code or instructions executable by one or more processors. Example tangible, computer-readable media may include, but is not limited to, volatile media, non-volatile media, removable media and non-removable media implemented in any method or technology for storage of information such as computer readable instructions, data structures, program modules or other data. Accordingly, external memory 134 can include random access memory (RAM), read-only memory (ROM), hard drive storage, temporary storage, non-volatile memory, flash memory, optical memory, or any other suitable memory for storing software objects and/or computer instructions. As an example, external memory 134 may be a flash drive, an external hard drive, a memory card (e.g., an SD card), a CD, a DVD, or even cloud-based memory. External memory 134 can include database components, object code components, script components, or any other type of information structure for supporting the various activities and information structures described in the present disclosure.

Remote device(s) 136 may be any additional devices that are remote (i.e., distinct) from controller 102. Example remote device(s) 136 can include remote servers, computing devices, user interfaces, etc. In some embodiments, remote device(s) 136 include at least one computing device having memory and one or more processors, functionally similar to, or the same as, the memory and processor(s) of controller 102 described below. In some embodiments, remote device(s) 136 include a smartphone, smartwatch, or personal computer associated with a user. In other words, controller 102 may transmit and receive (i.e., communicate) data with the user's personal computing device. In general, remote device(s) 136 can include, or may include at least one device having, a user interface. For example, a user's smartphone may include a touchscreen interface capable of displaying graphical images and/or receiving user inputs.

In this regard, a user interface may be any component or group of components that allows a user (e.g., a medical professional) to interact with controller 102. In some embodiments, the user interface includes at least a display screen, such as a liquid crystal display (LCD), LED display, or the like, which is configured to display graphics (i.e., images) and/or text. For example, the user interface may include an LCD that displays data relating to a user's biomechanical and/or biometric characteristics. In some embodiments, the user interface, and thereby remote device(s) 136, also includes one or more user input devices, such as buttons, keys, a keypad, a keyboard, a mouse, etc. In some such embodiments, such as in the example provided above, the user interface can include a touchscreen display that both presents information (e.g., displays unique visual symbols) and receives user inputs in the form of touches on the screen itself.

Still referring to FIG. 1, controller 102 is shown to include a processing circuit 104 which further includes a processor 106 and memory 110. Processor 106 an be a general-purpose processor, an application specific integrated circuit (ASIC), one or more field programmable gate arrays (FPGAs), a group of processing components, or other suitable electronic processing components. In some embodiments, processor 106 is configured to execute program code stored on memory 110 to cause controller 102 to perform one or more operations as described herein.

Like external memory 134, memory ∠can include one or more devices (e.g., memory units, memory devices, storage devices, etc.) for storing data and/or computer code for completing and/or facilitating the various processes described in the present disclosure. In some embodiments, memory 110 includes tangible, computer-readable media that stores code or instructions executable by processor 106. Tangible, computer-readable media refers to any media that is capable of providing data that causes controller 102 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 110 can include random access memory (RAM), read-only memory (ROM), hard drive storage, temporary storage, non-volatile memory, flash memory, optical memory, or any other suitable memory for storing software objects and/or computer instructions. Memory 110 can include database components, object code components, script components, or any other type of information structure for supporting the various activities and information structures described in the present disclosure. Memory 110 can be communicably connected to processor 106, such as via processing circuit 104, and can include computer code for executing (e.g., by processor 106) one or more processes described herein.

While shown as individual components, it will be appreciated that processor 106 and/or memory 110 can be implemented using a variety of different types and quantities of processors and memory. For example, processor 106 may represent a single processing device or multiple processing devices. Similarly, memory 110 may represent a single memory device or multiple memory devices. Additionally, in some embodiments, controller 102 may be implemented within a single computing device (e.g., one server, one housing, etc.). In other embodiments controller 102 may be distributed across multiple servers or computers (e.g., that can exist in distributed locations). For example, controller 102 may include multiple distributed computing devices (e.g., multiple processors and/or memory devices) in communication with each other that collaborate to perform operations.

In some embodiments, one or more of the functions of controller 102 described herein may be performed or implemented by remote device(s) 136. In other words, in such embodiments, controller 102 may merely collect data from sensor array 132 and remote device(s) 136 may process the collected data. This configuration (e.g., distributed computing via remote device(s) 136) may be beneficial in reducing the processing workload, and thereby processing power requirements, for controller 102. As such, controller 102 may be may small enough to be comfortable carried and/or worn by a user. Additionally, because controller 102 is generally battery powered, using remote device(s) 136 to perform some or most of the processing described below (e.g., with respect to memory 110) can allow for the battery capacity of controller 102 to be reduced (e.g., leading to a reduction in size and weight) and/or can result in a greater battery life; thus, a longer operating time between recharging or replacing the battery. It should be appreciated that all such configurations of controller 102, sensor array 132, and remote device(s) 136 are contemplated herein.

Memory 110 is shown to include a balance and motion analyzer 112 receive and analyze force data provided by sensor array 132 to determine various biomechanical characteristics for a user. As described above, and as described in greater detail below with respect to FIGS. 2A-2E, sensor array 132 may include a plurality of force sensors positioned under various parts of a user's foot (e.g., when worn in the user's shoe(s)). Thus, balance and motion analyzer 112 may receive force data from each sensor of sensor array 132 and may interpret the force data to determine an amount of downward force exerted by the user against the ground (e.g., or the sole of the shoe). In this manner, balance and motion analyzer 112 may determine and record, in real-time or near-real time (e.g., within one (1) msec of real-time), the ground reaction force as the users stands or moves.

In some embodiments, balance and motion analyzer 112 is also configured to calculate and/or track a center-of-pressure (CoP) for each of the user's feet based on the force data provided by sensor array 132. CoP may indicate which part of the foot the downward force exerted by the user while standing or moving is centered upon. By tracking CoP, balance and motion analyzer 112 can evaluate the user's movements and balance to identify abnormalities. For example, tracking the user's CoP during a specific movement (e.g., a squat) can identify if the user is placing too much force on specific areas of the foot. In a squat, for example, CoP may be useful in determining if the user is leaning too far forward or backward, which can indicate incorrect form. If abnormalities are detected, balance and motion analyzer 112 may generate a recommendation for the user to correct the abnormality. In some embodiments, balance and motion analyzer 112 select recommendations from a database. For example, balance and motion analyzer 112 may query a local or remote database that includes various known abnormalities (e.g., leaning too far forward during a squat, leaning back on heels when standing still, etc.) and may cross-reference an identified abnormality with known (i.e., predetermined) corrective actions.

In some embodiments, balance and motion analyzer 112 uses a machine learning algorithm (e.g., a neural network) to evaluate a user's body mechanics and movements. Specifically, force data from sensor array 132 may be fed into the machine learning algorithm and the algorithm may “learn” the user's balance and movements (e.g., typical CoP, CoP during certain movements, common movements, etc.). When a movement is detected that does not match a user's typical pattern, balance and motion analyzer 112 may determine that an abnormality is detected. As described herein, the machine learning algorithm may be any suitable algorithm, such as decision trees, random forest, neural networks, logistic regression, Support Vector Machine (SVM), etc., or the Gaussian Process Regression (GPR) model described below.

In some embodiments, balance and motion analyzer 112 can use the force data provided by sensor array 132 to determine a weight of the user and/or a load carried by the user. For example, balance and motion analyzer 112 may determine (e.g., using the force data) that the user is carrying additional weight, such as a backpack, and can provide the user with recommendation to lower the amount of carried weight (e.g., if the additional weight is over a threshold, which may be determined based on the user's weight) or to adjust the positioning of the weight to improve the user's posture. Further, the user's weight may be tracked over time to provide recommendations for weight loss, which can help to prevent obesity and reduce injury risk. Unlike traditional scales which typically require the user to weigh themselves and record the measured weight, system 100 may be utilized regularly (e.g., daily) and without additional input from the user.

In some embodiments, balance and motion analyzer 112 utilizes data from one or more IMUs included in sensor array 132 for calculating the user's center-of-mass (CoM). Specifically, the IMUs may provide angular rate and/or specific force data, collected as the user moves, which can be used to calculate the distance and angle between the IMUs (and thereby the user's feet). By combining the angular rate and/or specific force data from the IMUs with the force data from the one or more force sensors in sensor array 132, balance and motion analyzer 112 can calculate the user's CoM. Similar to CoP, the calculated CoM may then be used to identify abnormalities or inefficiencies in the user's posture and movements, which in turn can be used to provide the user recommendations for improved body mechanics.

Memory 110 is also shown to include a lower extremity angle predictor 114 configured to predict an angle of the user's lower extremities based on the force data collected by sensor array 132. “Lower extremities” may refer to any portion of the human body below the midsection; however, as used herein, “lower extremities” generally refers to the user's ankles, knees, hips, and lower back (e.g., up to the lumbosacral joint (L5-S1)). In some embodiments, lower extremity angle predictor 114 includes and implements a predictive model for predicting angles of each of the user's ankles, knees, hips, and lower back based on force data from sensor array 132. As described herein, the predictive model implemented by lower extremity angle predictor 114 may be any mathematical model but is generally a machine learning model.

In some such embodiments, the predictive model is a linear regression model, such as a non-parametric Bayesian regression learner or GPR model; although it will be appreciated that any other suitable machine learning model may be used (e.g., decision trees, random forest, neural networks, logistic regression, Support Vector Machine (SVM), etc.). In some embodiments, lower extremity angle predictor 114 may post process collected data from sensor array 132 to extract features (e.g., the outputs of each of the one or more force sensors included in sensor array 132), which are then fed into the predictive model to predict an angle of the user's ankle(s). Subsequently, the user's knee, hip, and lower back angle can be predicted. In some embodiments, the predict model relies on inverse dynamics, such that the predicted angled of a first joint (e.g., the ankle) below a second joint (e.g., the knee) will dynamically affect the second joint. In this regard, the predicted ankle angle will be used to predict a knee angle, the predicted knee angle will be used to predict the hip angle, and so on.

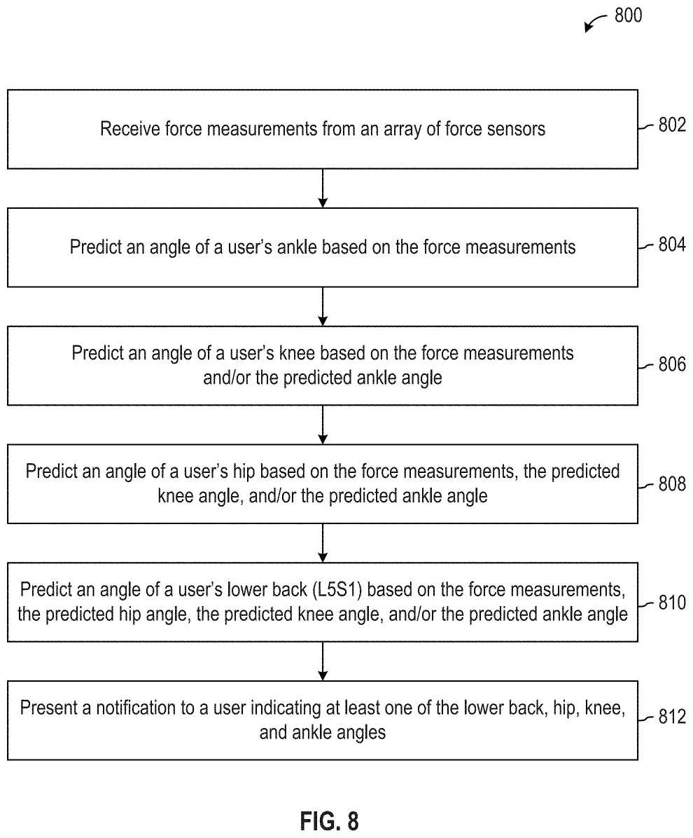

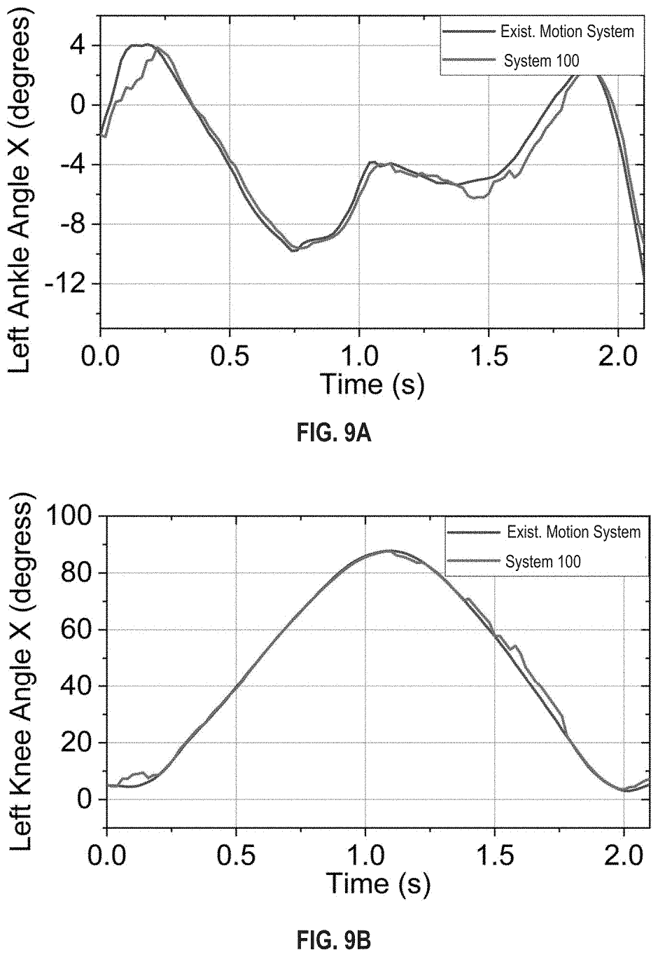

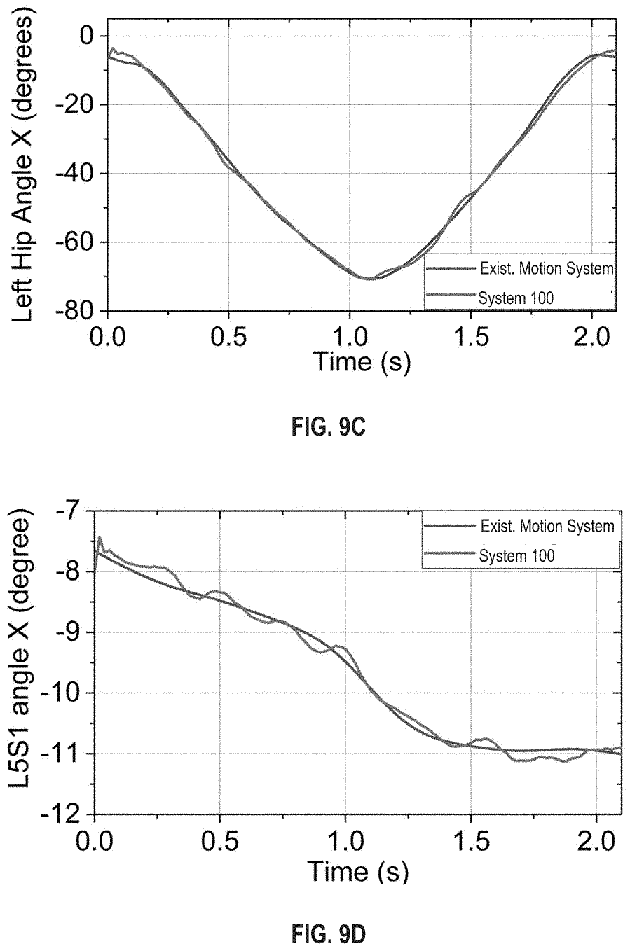

In some embodiments, the predictive model implemented by lower extremity angle predictor 114 is initiated (e.g., upon first use by a user) in a general form that can be adapted to a particular user. In other words, the predictive model may learn, or be trained, for the specific user. For example, weights may be applied to one or more variables of the predictive model, and the values of the weights may be modified to adapt the model to an individual user (e.g., based on data collected over time for the user). Various features and advantages of lower extremity angle predictor 114 are described in greater detail below with respect to FIGS. 8 and 9A-9D.

Memory 110 is also shown to include a gait analyzer 116 configured to detect and analyze a user's gait pattern. In some embodiments, gait analyzer 116 receives force data and/or resonant frequency data from sensor array 132. Specifically, force data may be received from one or more force sensors of sensor array 132, while resonance data may be received from an antenna of sensor array 132. For example, the antenna may measure a resonant frequency of the user's foot/feet during a gait cycle (e.g., over the course of one or two steps) to detect variations (i.e., changes) in the foot's resonant frequency. Based on the force and/or resonant frequency data, the user's gait patten can be determined. In some embodiments, gait analyzer 116 can evaluate the user's gait to identify abnormalities (e.g., heel-striking when running, toe walking, inefficient or harmful weight distribution, etc.) and/or to provide recommendations for an improved gait. Using only force data from sensor array 132, for example, the user's gait may be evaluated to learn patterns of force across the array, which may indicate the user's gait pattern for a “walk cycle.” For example, force data may be evaluated to determine whether the user walks on their heels, flat footed, etc., number of steps per second, and other gait metrics. In some embodiments, a gait pattern is detected and evaluated by any of the machine learning models described above.

Still referring to FIG. 1, memory 110 is also shown to include an environmental monitor 118. Environmental monitor 118 may be configured to receive data from one or both of a temperature sensor and a moisture sensor included in certain configurations of sensor array 132 (e.g., as shown in FIG. 2D). Specifically, due to the placement of sensor array 132 in the user's shoe, the temperature sensor may measure a temperature with the shoe and the moisture sensor may measure a humidity (i.e., moisture) level within the shoe. As those in the art will appreciate, fungal infections of the foot are directly related to high temperature and/or humidity levels within a user's shoe(s). Thus, environmental monitor 118 may track temperature and/or humidity levels to identify high-risk situations, such as when the temperature and/or humidity within the shoe exceeds a predefined threshold for a predetermined amount of time. If a high-risk situation (e.g., for the development of a fungal infection, such as athlete's foot) is detected, environmental monitor 118 may cause controller 102 to present a notification (e.g., via a user interface, such as the user interface included in remote device(s) 136) to the user prompting the user to remove their shoes, change their socks, etc.

Additional or alternately, memory 110 can include a biometric monitor 120. Biometric monitor 120 may be configured to receive data from any of a heart rate sensor, an O₂ sensor, or a bioimpedance sensor included in certain configurations of sensor array 132 (e.g., as shown in FIG. 2C). The heart rate sensor, O₂ sensor, and/or bioimpedance sensor may provide biometric monitor 120 with measurements of the user's heart rate, oxygen saturation, and body composition, which are generally referred to herein as “biometric characteristics.” In this manner, biometric monitor 120 may track the user's biometric characteristics. For example, biometric monitor 120 can monitor and track the user's biometric characteristics (e.g., heart rate and oxygen saturation) while the user performs an exercise, to provide the user with biometric feedback, to calculate calories burned, etc. Additionally, in some embodiments, bioimpedance data, which can indicate the user's body composition, may be tracked and evaluated to provide the user with recommendations for increasing or decreasing body fat (e.g., in combination with weight tracking, as described above with respect to balance and motion analyzer 112).

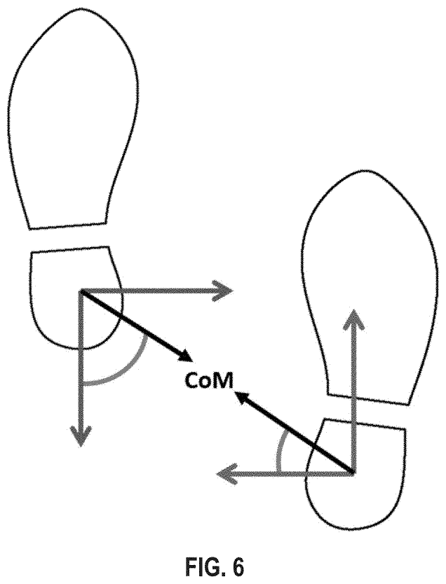

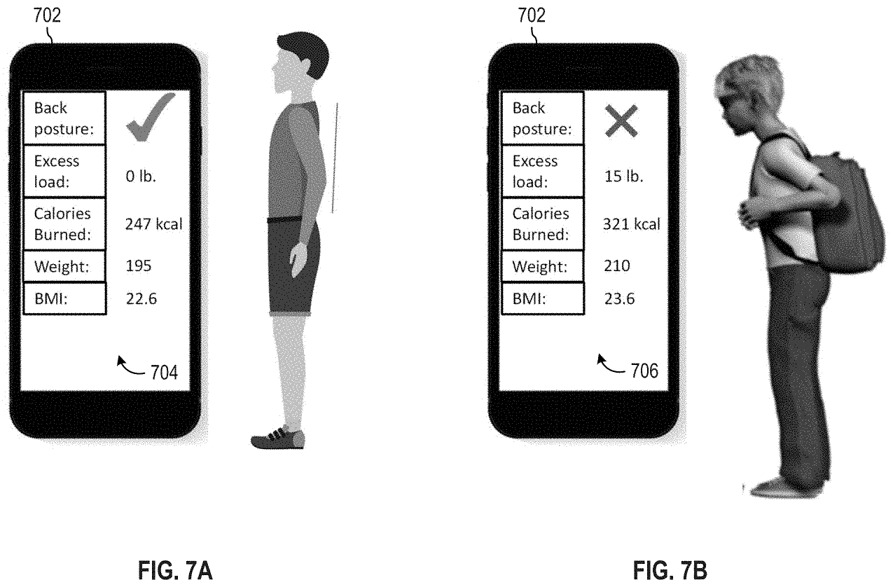

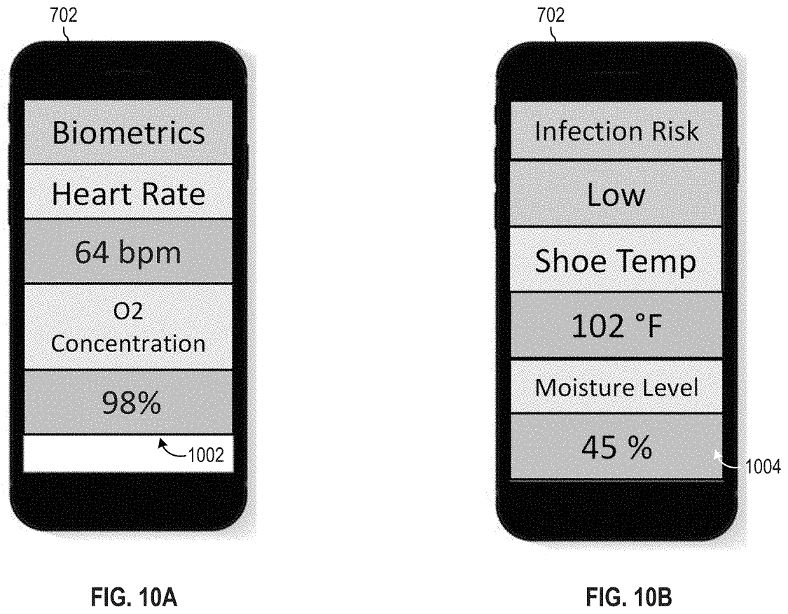

In some embodiments, memory 110 includes a user interface (UI) generator 122, which is configured to generate graphical user interfaces (GUIs). In some embodiments, the GUIs generated by UI generator 122 are displayed on a screen or other interface of controller 102 (not shown) or that is communicably coupled to controller 102. In some embodiments, UI generator 122 may transmit generated GUIs or data relating to GUIs to remote device(s) 136 for display. As mentioned above, for example, remote device(s) 136 may include a user's smartphone or smartwatch, which may be configured to display GUIs generated by UI generator 122. Example GUIs that may be generated by UI generator 122 are shown in FIGS. 7A, 7B, 10A, and 10B; however, it will be appreciated that UI generator 122 may be configured to generate any number of additional user interfaces for providing feedback regarding the biomechanical, biometric, and environmental characteristics associated with a user. For example, UI generator 122 may generate GUIs for providing the user with recommendations to correct motion or balance abnormalities, to improve gait, to reduce or increase weight or body fat, to adjust an amount or placement of carried (e.g., additional) weight, etc.

Referring now to FIGS. 2A-2E, diagrams illustrating various configurations of sensor array 132 are shown, according to some embodiments. Turning first to FIG. 2A, a first configuration of sensor array 132 is shown having a plurality of force sensors 202-212 positioned on a flexible substrate 214. Example types of force sensors 202-212 include, but are not limited to, force sensing resistors (FSRs), piezoresistive (PZR) sensors, capacitive force sensors, and fabric-based strain sensors. However, it will be appreciated that force sensors 202-212 may be any suitable type of sensors for measuring force or pressure.

As mentioned above, flexible substrate 214 may be configured to be placed under an insole of the user's shoe(s) or to replace the insole of the user's shoe(s). Accordingly, flexible substrate 214 may be formed of any flexible material, preferably suitable for handling the repeated friction of a user's foot moving in the shoe (e.g., when walking). Example flexible materials for flexible substrate 214, but are not limited to, foam rubber, cellular polymers, latex, cork, any flexible plastic (e.g., acrylic, polycarbonate, polyethylene, ABS, etc.), etc. In some embodiments, flexible substrate is a flexible printed circuit board (PCB) material, such as a polyimide double sided copper clad laminate (e.g., DuPont™ Pyralux®. In some embodiments, flexible substrate 214 reinforced with a flexible polymer, such as polycarbonate or polyethylene. In some embodiments, flexible substrate 214 may include a layer of cellulose acetate to protect force sensors 202-212 and any wires used to couple force sensors 202-212 to controller 102.

As also mentioned above, force sensors 202-212 may be configured to measure an amount of force, or downward pressure, exerted by a user (e.g., a wearer of shoe into which sensor array 132 is installed) against the ground. In some embodiments, sensor array 132 includes six force sensors 202-212, additionally labeled in FIG. 2A as S₁-S₆. Each of force sensors 202-212 may be positioned under a common pressure point of a user's foot. However, in other embodiments, sensor array 132 includes at least three force sensors. In particular, at least one sensor may be positioned on a heel portion of flexible substrate 214 (e.g., force sensors 202, 204), at least sensor may be positioned on a mid-foot (i.e., ball) portion of flexible substrate 214 (e.g., force sensors 206-210), and at least one sensor may be positioned on a toe portion of flexible substrate 214 (e.g., force sensor 212). In other words, at least one of each of force sensors 202-212 may be positioned on flexible substrate 214 to be under the heel, the ball, and a toe of the user's foot, when installed in a shoe.

In some embodiments, sensor array 132 further includes an IMU 220 disposed on flexible substrate 214. In some such embodiments, IMU 220 is positioned on flexible substrate 214 to be under the user's heel or towards the heel portion of flexible substrate 214, as shown in FIG. 2B. As mentioned above, IMU 220 may be configured to detect one or more the specific force, angular rate, and orientation of the user's shoe (and thereby the user's foot), which may be utilized by controller 102 to calculating the user's CoM and can also improve or supplement an analysis of the user's gait. Placement of IMU 220 in the heel of a shoe (e.g., when sensor array 132 is installed in the shoe), in particular, can improve measurements for calculating CoM. For example, the heel of the shoe may be the best reference position for IMU 220 due to the distance between a user's toes and heel, which increases the resolution of detection for calculating CoM and/or CoP.

In some embodiments, sensor array 132 further includes a biometric sensor array 222, also referred to as combined biometric sensor 222, as shown in FIG. 2C. Biometric sensor 222 is shown to be disposed on flexible substrate 214 so as to be positioned under a lateral portion of the user's foot when in use. Biometric sensor 222 is configured to measure or detect the user's biometric characteristics, including at least one of the user's heart rate, oxygen saturation, and bioimpedance. This biometric data can provide the user with a more complete picture of their overall health and can be used to track user workouts with requiring the user to use additional sensors (e.g., a heart rate monitor) or activity tracking devices (e.g., a smartwatch). Further, heart rate data may be used to supplement activity data collected by force sensors 202-212 and/or IMU 220 (e.g., number of steps, stride length, steps per minute, etc.) to provide more accurate calculations of calories burned, amount of sweat lost, etc.

In some embodiments, sensor array 132 further includes a temperature sensor 224, as shown in FIG. 2D. Temperature sensor 224 is shown to be disposed on flexible substrate 214 and may, in some embodiments, be positioned under the user's arch; however, other positions of temperature sensor 224 are contemplated herein. Temperature sensor 224 may be configured to measure a temperature of the user's foot and/or a temperature within the user's shoe. In some embodiments, either in addition to temperature sensor 224 or in a configuration without temperature sensor 224, sensor array 132 also includes a moisture sensor 226. In some such embodiments, moisture sensor 226 may be positioned near or under the user's toes (i.e., in a toe portion of flexible substrate 214), as the toe-box region of a shoe may typically experience the greatest humidity levels. As mentioned above, data from temperature sensor 224 and moisture sensor 226, either alone or in combination, can be used to detect high-risk environments that may promote the growth of harmful fungus or bacteria (e.g., athlete's foot). If a high-risk environment is detected, the user may be warned (e.g., by a notification provided by controller 102 to a user interface of remote device(s) 136) so that they might mitigate the risk by removing their shoe(s), changing socks, etc.

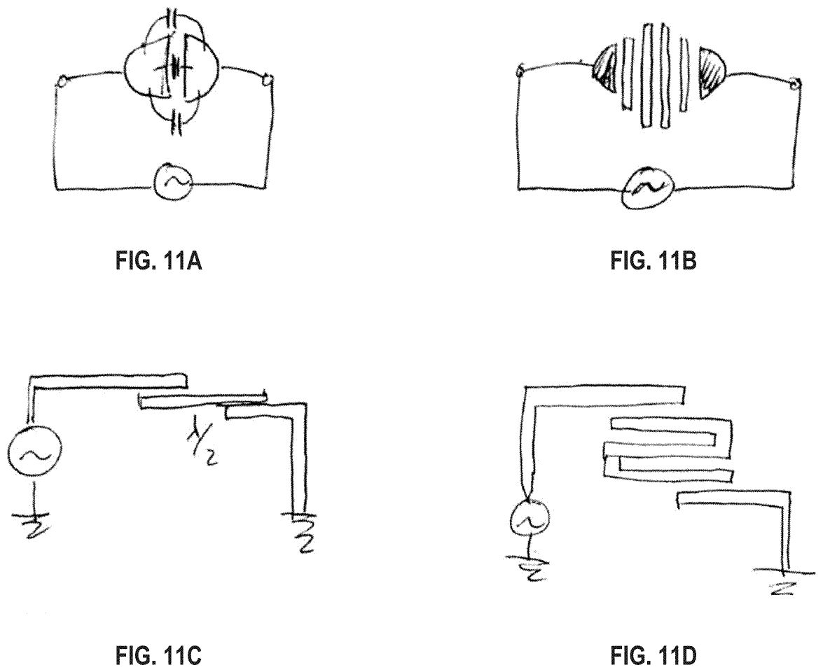

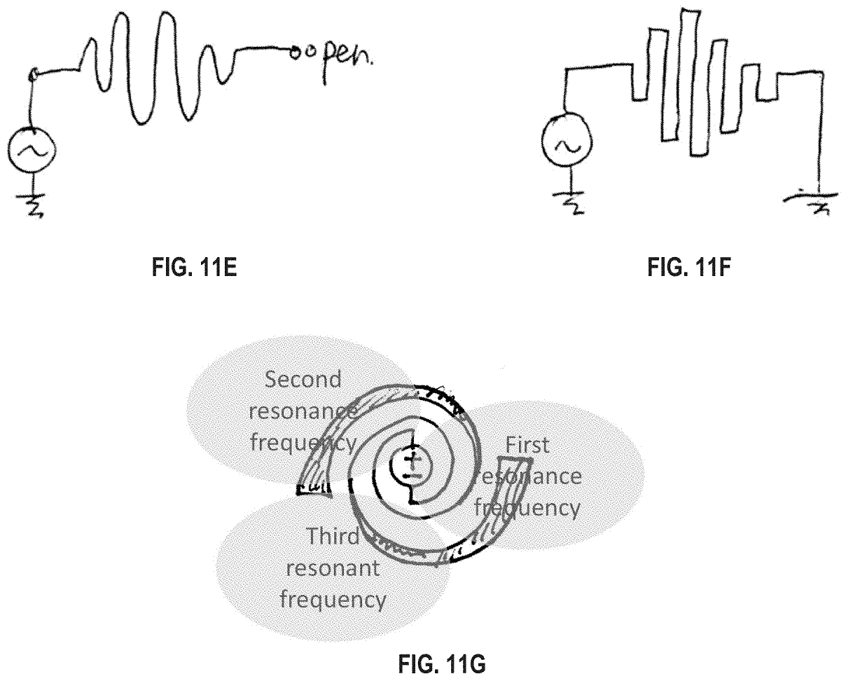

In some embodiments, sensor array 132 further includes an antenna 228, which may be positioned near a heel portion of flexible substrate 214, as shown in FIG. 2E. Antenna 228 may be configured to measure a resonant frequency of the user's foot, particularly during a gait cycle. By measuring changes in the foot's resonant frequency, controller 102 may be able to accurately profile the user's gait pattern. In some embodiments, the gait pattern determined from resonant frequency data provided by antenna 228 may be further supplemented with data from force sensors 202-212 and/or IMU 220, as described above. Various configurations of antenna 228 are shown in FIGS. 11A-11G, as described in greater detail below. In some embodiments, antenna 228 can replace force sensors 202-212 on flexible substrate 214, such that antenna 228 is solely used to detect the ground reaction (e.g., downward) force applied by a user based on the resonant frequency of the user's foot throughout a gait cycle or during a movement. In some embodiments, multiple antennas 228 may be positioned on flexible substrate 214 in place of force sensors 202-212.

In general, force sensors 202-212, IMU 220, biometric sensor 222, temperature sensor 224, moisture sensor 226, and antenna 228 are communicably coupled to controller 102 via a direct, wired connection (e.g., via a wire or cable). However, in some embodiments, one or more of force sensors 202-212, IMU 220, biometric sensor 222, temperature sensor 224, moisture sensor 226, and antenna 228 are communicably coupled to controller 102 via an indirect connection (e.g., through a separate controller) and/or via a wireless connection. In some embodiments, one or more of force sensors 202-212, IMU 220, biometric sensor 222, temperature sensor 224, moisture sensor 226, and antenna 228 are active sensors that require a power source. Accordingly, in some such embodiments, controller 102 may provide power to any of the above-identified sensors.

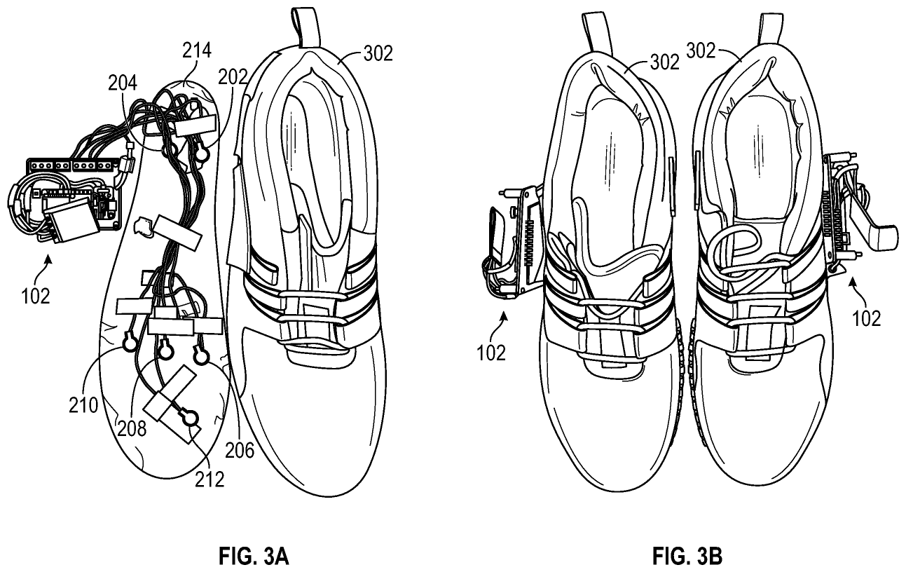

Referring now to FIGS. 3A and 3B, an example implementation of system 100 and sensor array 132 is shown, according to some embodiments. In FIGS. 3A, for example, sensor array 132 is configured for a right shoe 302. More specifically, sensor array 132 is positioned on flexible substrate 214, which is intended to replace a standard insole for shoe 302. Coupled to sensor array 132 is controller 102, which may be positioned within shoe 302 (e.g., in the sole or tongue of shoe 302) or, as shown, on an exterior of shoe 302. FIG. 3B shows a pair of shoes 302 each having a sensor array 132 positioned therein and an attached controller 102. It will be appreciated that the form factor of controller 102 shown in FIGS. 3A and 3B is merely representative and that controller 102 may be configured in a smaller and more compact format. In some embodiments, components of controller 102 and/or controller 102 itself may be embedded into the shoe (e.g., in the sole, tongue, sidewalls, etc.). In embodiments where controller 102 is configured only to collect data from sensor array 132 and transmit it to a user's device (e.g., remote device(s) 136) for processing, controller 102 may be compact enough to be disposed on flexible substrate 214.

Body Mechanic Detection

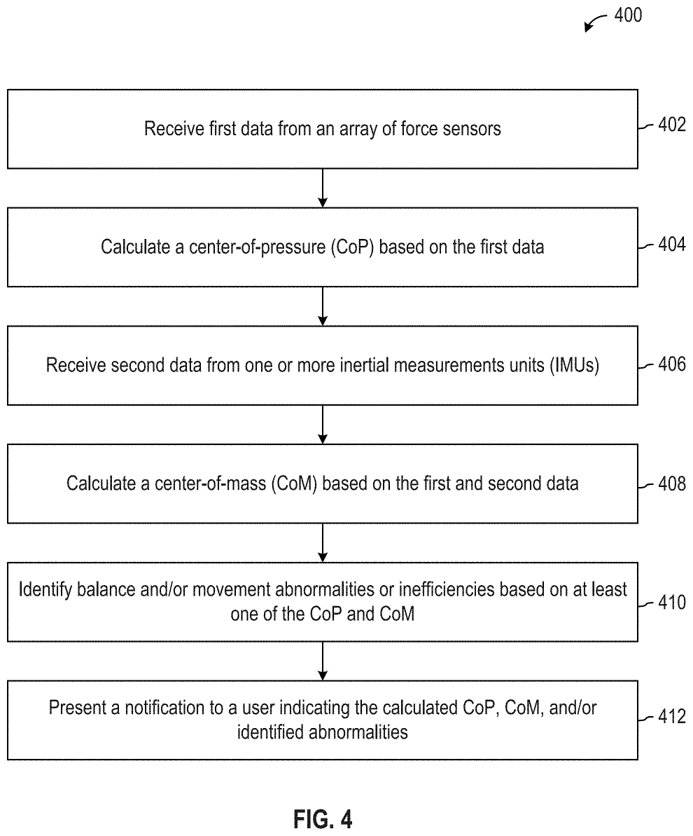

Referring now to FIG. 4, a flow diagram of a process 400 for determining various body mechanics of a user using system 100 is shown, according to some embodiments. Process 400 may be implemented by controller 102, for example, as described above; however, it will be appreciated that, in embodiments where controller 102 only collects data from sensor array 132, process 400 may be implemented by a remote computing device, such as remote device(s) 136. It will be appreciated that certain steps of process 400 may be optional and, in some embodiments, process 400 may be implemented using less than all of the steps. Additionally, it will be appreciated that the steps of process 400 are not necessarily implemented in the order shown.

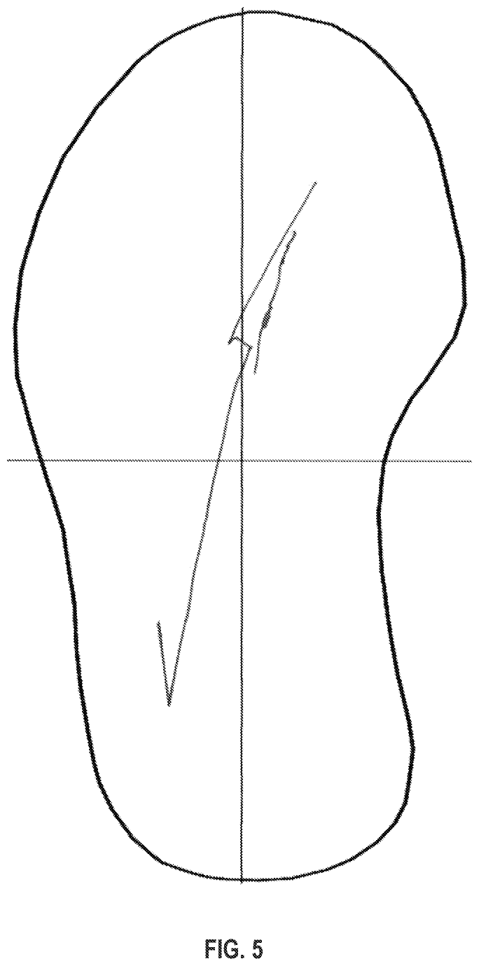

At step 402, first data is received from an array of force sensors. In particular, the array of force sensors may be force sensors 202-212 of sensor array 132; however, the array of force sensors typically includes at least three force sensors positioned on a flexible substrate (e.g., flexible substrate 214) configured to be placed in the user's shoe, including at least one sensor positioned under each of the heel, ball, and toe of the user's foot. Accordingly, the first data may be a measured force exerted by the user against the ground (i.e., a pressure) as the user stands and/or moves. Using the first data, at step 404, a CoP can be calculated for one or both the user's feet. As described in greater detail below with respect to FIG. 5, CoP may indicate an area of the foot on which the greatest amount of force is focus, which in turn can indicate the user's body position and/or movements. Further, CoP can be tracked for one or both feet to monitor user movements.

At step 406, second data may be received from one or more IMUs (e.g., IMU 220). As discussed above, data from IMU 220, for example, may be used to determine a position of each of the user's feet (e.g., in relation to one another). Further, IMU 220 may detect movement of the user's feet. Using the angular rate, specific force, and/or orientation data provided by IMU 220, at step 408, the user's CoM can be calculated. In some embodiments, CoM is calculated based on IMU data in combination with the force data collected at step 402. As described in greater detail below with respect to FIG. 6, the user's CoM may not only be useful in evaluating the user's movements (e.g., gait patterns) but may also be used to determine whether the user is carrying weight (e.g., a backpack, a box, etc.) inefficiently.

At step 410, balance and/or movement abnormalities and/or inefficiencies are identified based on at least one of the user's CoP and CoM. For example, tracking the user's CoP and/or CoM during a specific movement, such as a squat, can identify if the user is placing too much force on specific areas of the foot. In this example, a CoP centered on the front of the user's foot may indicate that the user is leaning too far forward during the squat, which can indicate incorrect form and can cause injuries. To continue another example provided above, a CoM that is centered too far forwards or backwards may indicate that the user is carrying weight inefficiently, which may also lead to injuries.

At step 412, a notification may be presented to the user that indicates the calculated CoP, the calculated CoM, and/or any identified abnormalities or inefficiencies. In some embodiments, the notification is displayed by a user interface of system 100 (not shown) or is transmitted by controller 102 to a remote device having a user interface (e.g., remote device(s) 136). The notification, for example, may simply indicate the CoP of one or both feet, and/or may indicate the user's CoM, in a graphical format. In some embodiments, the notification indicates whether the user's CoP and/or CoM indicate any abnormalities or inefficiencies. If abnormalities or inefficiencies are detected, the notification may further include a recommendation for the user to correct the abnormality or inefficiency. For example, a recommendation may be selected from a database that includes various known abnormalities (e.g., leaning too far forward during a squat, leaning back on heels when standing still, etc.). Specifically, in such embodiments, the database may include cross-references between identified abnormalities and efficiencies and known (i.e., predetermined) corrective actions. In some embodiments, the notification may include a selectable link that navigates the user to a webpage or software application for providing additional information about the user's movements, detected abnormalities, etc. For example, the selectable link may include videos that demonstrate proper form for various movements.

Referring now to FIG. 5, an example diagram illustrating CoP detection using system 100 is shown, according to some embodiments. As mentioned above, CoP may indicate an area of the foot on which the greatest amount of force is focused, which in turn can indicate the user's body position and/or movements (e.g., leaning forward onto toes, leaning back onto heels). Further, CoP can be tracked for one or both feet to monitor user movements. Illustrating CoP graphical, as in FIG. 5, can help the user to easily identify trends and abnormalities in their movements. In the example of FIG. 5, CoP (e.g., shown as a line 502) was tracked for the left foot of a user while the used performed a squat. As shown, the user briefly placed a majority of their weight on their heels and transitioned their weight forward, towards their toes. In some cases, this may indicate that the user is placing too much weight on the front or back of their foot when at the bottom portion of a squat, which can indicate incorrect form.

In some embodiments, CoP is calculated based on the response from one or more of force sensors 202-212. In such embodiments, a resistance versus force curve (e.g., mapping measured resistance to a force value) is used to determine an amount of force applied to a given one of force sensors 202-212. Force, or pressure, can then be determined based on the physical dimensions of the force sensor(s). Subsequently, the calculated force is applied to a sum of forces (i.e., pressures) equation based on the positions of force sensors 202-212 on flexible substrate 214. In some embodiments, CoP is calculated using the equation listed below.

$${\hat{x}}_{CoP} = \frac{{\sum }_{n = 1}^{N} {p}_{n} {x}_{n}}{{\sum }_{n = 1}^{N} {p}_{n}} ,$$

$${\hat{y}}_{CoP} = \frac{{\sum }_{n = 1}^{N} {p}_{n} {y}_{n}}{{\sum }_{n = 1}^{N} {p}_{n}}$$

$$\vec{CoP} = ( {\hat{x}}_{CoP} , {\hat{y}}_{CoP} )$$

Referring now to FIG. 6, an example diagram illustrating CoM detection using system 100 is shown, according to some embodiments. CoM may be calculated based, in part, on data provided by IMU 222. In particular, a distance and angle between two or more IMUs, one placed in each shoe of the user (e.g., on separate sensor arrays 132), may be measured based on IMU data. CoM may then be calculated based on the determined distance and angle between the IMUs. Based on the CoM, various characteristics of a user's movements can be determined. For example, it can be determined if the user is leaning too far forward or backward during certain movements, or whether the user has incorrectly distributed a carried weight.

In some embodiments, CoM is calculated based, at least in part, on the calculated CoP, as shown in FIG. 5. As mentioned above, CoP may indicate a center point of each foot of a user and/or a point where the force applied by the user against the ground is centered. Thus, a 2-dimensional (2D) CoM may be calculated based on the distance between the user's shoes (e.g., between two of sensor arrays 132) and the angle between the shoes (e.g., based on IMU data). However, it should be appreciated that the CoM calculation may be dependent on ensuring that the user is not resting their body on any external objects. In some embodiments, CoM is calculated using the equation listed below.

$${\hat{X}}_{CoM} = \frac{{x}_{{CoP}_{L}} + {x}_{{CoP}_{R}}}{2} ,$$

$${\hat{Y}}_{CoM} = \frac{{y}_{{CoP}_{L}} + {y}_{{CoP}_{R}}}{2}$$

$$\vec{CoM} = ( {\hat{Y}}_{CoM} , {\hat{Y}}_{CoM} )$$

Referring now to FIGS. 7A and 7B, example user interfaces 704, 706 for providing feedback regarding a user's body mechanics are shown, according to some embodiments. In this example, both interfaces 704, 706 are presented on a user device 702, which may be a user's smartphone; however, more generally, user device 702 is one of remote device(s) 136. Interfaces 704, 706 both indicate a plurality of biomechanical and biometric data for the user. For example, each of interfaces 704, 706 indicate a number of calories burned by the user over a time period (e.g., during an exercise, over the course of a day, etc.), a current weight of the user (e.g., detected by sensor array 132), and a body mass index (BMI) of the user. As also shown, interfaces 704, 706 both indicate an amount of additional weight (i.e., excess load) carried by the user, which may be calculated by subtracting the user's measured weight from a known (e.g., previously measured) weight.

In interface 704, the user is shown to be carrying zero additional pounds and it is determined (e.g., by system 100) that the user is maintaining correct posture. In contrast, interface 706 shows that the user is carrying 15 extra pounds and that the user is not maintaining correct posture (e.g., leaning forward to offset the weight of a backpack). In some embodiments, a notification may be displayed on or over interfaces 706 that provides the user with a recommendation for correcting their posture, such as reducing the carried weight or adjusting the positioning of the weight.

Lower Extremity Angle Detection

Referring now to FIG. 8, a flow diagram of a process 800 for predicting the positioning (e.g., angle) of a user's lower extremities using system 100, according to some embodiments. Process 800 may be implemented by controller 102, for example, as described above; however, it will be appreciated that, in embodiments where controller 102 only collects data from sensor array 132, process 800 may be implemented by a remote computing device, such as remote device(s) 136. It will be appreciated that certain steps of process 800 may be optional and, in some embodiments, process 800 may be implemented using less than all of the steps.

Additionally, it will be appreciated that the steps of process 800 are not necessarily implemented in the order shown.

At step 802, force measurements are received from an array of force sensors (e.g., force sensors 202-212 of sensor array 132). In particular, the array of force sensors may be force sensors 202-212 of sensor array 132; however, the array of force sensors typically includes at least three force sensors positioned on a flexible substrate (e.g., flexible substrate 214) configured to be placed in the user's shoe, including at least one sensor positioned under each of the heel, ball, and toe of the user's foot. Accordingly, the first data may be a measured force exerted by the user against the ground (i.e., a pressure) as the user stands and/or moves at each of force sensors 202-212, or any subset of force sensors 202-212.

At step 804, an angle of the user's ankle is predicted based on the force measurements. As mentioned above with respect to FIG. 1, a predictive model may be implemented to predict the angle of the user's ankle (e.g., or ankles, when two shoes containing system 100 are worn by a user). In some embodiments, the predictive model is a supervised non-parametric Bayesian regression learner, Gaussian Processes Regression (GPR); however, any other suitable predictive (e.g., machine learning) model may be selected, including those listed above. Advantageously, the computational cost of predicting lower extremity angles using the GPR or other, similar predictive models is low, allowing for the use of low cost and/or low energy processors (e.g., microcontrollers). In some embodiments, prior to predicting the ankle angle, the force measurements received at step 802 are evaluated to extract features, which are fed into the predictive model. For example, to predict an ankle angle, the outputs of force sensors 202-212 may be extracted and provided as inputs to the predictive model, which may output a predicted ankle angle based on the force measurements.

At step 806, an angle of the user's knee is predicted based on the force measurements and/or the predicted ankle angle. More specifically, in some embodiments, the knee angle is predicted by applying the force measurements received at step 802 and the predicted ankle angle from step 804 to the predictive model or to a second predictive model designed to predict knee angle(s). In this regard, the knee angle and any subsequent lower extremity joint angles may be predicted based on inverse dynamic, or the idea that the ankle of a first joint (e.g., the ankle) below a second joint (e.g., the knee) will impact the predicted angle of the second joint. Accordingly, the predictive model may output a predicted knee angle in degrees.

At step 808, an angle of the user's hip is predicted based on the force measurement, the predicted ankle angle, and/or the predicted knee angle. In some embodiments, the hip angle is predicted by applying the force measurements received at step 802, the predicted ankle angle from step 804, and the predicted knee angle from step 806 to the predictive model or to a third predictive model designed to predict hip angle(s). Accordingly, the predictive model may output a predicted hip angle in degrees.

Similarly, at step 810, an angle of the user's lower back is predicted based on the force measurement, the predicted ankle angle, the predicted knee angle, and/or the predicted hip angle. More specifically, an angle of the L5-S1 region of the user's lower back is predicted. In some embodiments, the lower back angle is predicted by applying the force measurements received at step 802, the predicted ankle angle from step 804, the predicted knee angle from step 806, and the predicted hip angle from step 808 to the predictive model or to a fourth predictive model designed to predict lower back angles. Accordingly, the predictive model may output a predicted lower back angle in degrees.

At step 812, a notification may be presented to the used that indicates one or more of the predicted lower extremity angles. In some embodiments, the notification is displayed by a user interface of system 100 (not shown) or is transmitted by controller 102 to a remote device having a user interface (e.g., remote device(s) 136). The notification, for example, may simply indicate the various predicted lower extremity angles in a graphical format, such as the graphs shown in FIGS. 9A-9D. In some embodiments, the notification indicates whether the predicted lower extremity angles indicate any abnormalities or inefficiencies. If abnormalities or inefficiencies are detected, the notification may further include a recommendation for the user to correct the abnormality or inefficiency. For example, a recommendation may be selected from a database that includes various known abnormalities (e.g., inefficient or possibly harmful knee position during a movement, poor posture, etc.). Specifically, in such embodiments, the database may include cross-references between identified abnormalities and efficiencies and known (i.e., predetermined) corrective actions. In some embodiments, the notification may include a selectable link that navigates the user to a webpage or software application for providing additional information about the user's movements, detected abnormalities, etc. For example, the selectable link may include videos that demonstrate proper form for various movements.

Referring now to FIGS. 9A-9D, example graphs showing the prediction of various lower extremity angles are shown, according to some embodiments. In particular, FIGS. 9A-9D show the prediction of an example user's ankle, knee, hip, and lower back angles as the user performs a squat. Additionally, each of FIGS. 9A-9D show a predicted joint angle in comparison with a know joint angle, as determined by a state-of-the-art motion tracking system, which requires a plurality of motion tracking orbs or markers to be positioned at various points on the user's body and which are tracked by a system of cameras. Thus, it can clearly be seen in FIGS. 9A-9D that system 100 is highly accurate in predicting the angles of a user's lower extremities. Taking FIG. 9A, for example, the left ankle angle of a user is tracked by both the state-of-the-art motion tracking system and system 100 over a period of time. During a squat, it can be seen that the user's angle briefly moved 4° from a starting position (e.g., standing upright, such that the user's ankle is perpendicular to the ground), moving to about −10° for a brief time, and returning towards the 4° mark. Here, it can clearly be seen that the angle predicted by system 100 closely tracks the angle detected by the state-of-the-art motion tracking system.

Biometric Detection

Referring now to FIGS. 10A and 10B, example user interfaces for providing feedback regarding a user's biometric characteristics are shown, according to some embodiments. In particular, FIG. 10A shows a biometric interface 1002 displayed on user device 702. Interface 1002 is shown to include various biometric data (e.g., collected by biometric sensor 222), including at least a current heart rate of the user (e.g., 64 bpm) and a current oxygen saturation (O₂) for the user (e.g., 98%). In some embodiments, interface 1002 may also display information such as a predicted body fat percentage for the user (e.g., based on bioimpedance data), a number of calories burned during a period of time (e.g., due to exercise), and any other biometric data.

FIG. 10B shown an environmental interface 1004 that indicates a temperature within the user's shoe (e.g., determined by temperature sensor 224) and a moisture (i.e., humidity) level within the user's shoe (e.g., determined by moisture sensor 226). Additionally, in some embodiments, interface 1004 may indicate a risk level for the user based on the environmental data. Specifically, the risk level may indicate the risk of fungal or bacterial growth in the user's shoe (e.g., on the user's foot) based on the environmental data. For example, high moisture and/or temperature levels, particularly for an extended period of time, may cause the user's risk level to increase (e.g., between low, medium, and high). In FIG. 10B, the user is shown to be at low risk for infection. However, if the user were determined to be at a high risk level, interface 1004 may also provide the user with recommendations for lowering the risk level (e.g., changing socks, removing their shoes for a period of time, etc.).

Antenna Geometries for Gait Detection

Referring now to FIGS. 11A-11G, example diagrams illustrating various configurations of antenna 228 are shown, according to some embodiments. Turning first to FIGS. 11A-11D, in general, various coupled antenna configurations are shown. A coupled antenna generally includes two or more conductors that are separated by a small gap to allow the electric fields of each of the conductors to couple. In sensor array 132, in particular, the conductors of antenna 228 may be positioned on a pressure point, such as the user's heel. Turning first to FIG. 11A, a first coupled antenna configuration is shown having two conductors that are coupled directly. In FIG. 11B, two conductors are coupled to a radiofrequency (RF) source and are separated by a plurality of additional conductors that are electromagnetically coupled. In FIG. 11C, two conductors are attached to an RF source and are separated by a third conductor. In some embodiments, the third conductor is configured to be half of a wavelength long. In FIG. 11D, two conductors are attached to an RF source and are separated by one or more additional conductor that are electromagnetically coupled.

Turning now to FIGS. 11E and 11F, two meandered antenna configurations are shown. In some embodiments, a radiating element of each of the antenna configurations shown in FIGS. 11E and 11F is meandered to resonate at the operating frequency. (e.g., 2.4 GHz or 5.8 GHz). Similar to the conductors of FIGS. 11A-11D, the meandered lines are mostly populated on the critical pressure point in a shoe. In FIG. 11E, a configuration is shown in which a conductive line is meandered to make a resonance at a pressure point (e.g., open circuit). In FIG. 11F, a meandered conductive line is formed with sharp (i.e., squared) corners to provide a more compact design; however, a resonance may still be formed at a pressure point of sensor array 132. In this example, the line is short-circuited.

Finally, in FIG. 11G, a spiral antenna configuration is shown. The spiral antenna may also be positioned at a pressure point of sensor array 132 (e.g., at the user's heel) and the resonant frequency of the spiral antenna may be tuned based on the positioning under the user's foot. Advantageously, multiple resonant frequencies may be detected. For example, a first resonant frequency is located at 5.5 GHz and the second resonant frequency is shifted to 5.9 GHz.

Example Implementations

Various example implementations of the present disclosure include:
A. A system for detecting human motion and body balance, the system comprising:
   a flexible substrate configured to be positioned in a shoe of a user;
   a sensor array comprising one or more force sensors positioned on the flexible substrate; and
   a controller communicably coupled to the sensor array, the controller configured to:
      receive force data from the sensor array;
      determine biomechanical characteristics for the user based on the force data, the biomechanical characteristics including at least one of a center-of-pressure (CoP), a center-of-mass (CoM), or lower extremity angles for the user; and
      provide an indication of the biomechanical characteristics to the user.
B. The system of example implementation A, wherein providing the indication of the biomechanical characteristics to the user comprises transmitting, by the controller, data including the biomechanical characteristics to a user device associated with the user to cause the user device to display a graphical user interface (GUI).
C. The system of example implementation A or B, the controller further configured to detect an abnormality in the biomechanical characteristics for the user.
D. The system of example implementation C, wherein the indication further includes a recommendation for correcting the abnormality.
E. The system of any one of example implementations A-D, wherein the lower extremity angles include a predicted angle for each of an ankle, a knee, a hip, and a lower back of the user.
F. The system of any one of example implementations A-E, wherein determining the biomechanical characteristics for the user comprises calculating the lower extremity angles using a machine learning model.
G. The system of any one of example implementations A-F, wherein the force data is provided as an input to the machine learning model, the machine learning model configured to predict an angle for each of an ankle, a knee, a hip, and a lower back of the user.
H. The system of any one of example implementations A-G, the one or more force sensors comprising force sensing resistors (FSRs), piezoresistive (PZR) sensors, capacitive force sensors, or fabric-based strain sensors.
I. The system of any one of example implementations A-H, wherein the flexible substrate is an insole of the shoe or wherein the flexible substrate is configured to be positioned under an insole of the shoe.
J. The system of example implementation I, the one or more force sensors comprising at least three sensors, wherein:
   a first subset of the one or more force sensors is positioned on the flexible substrate to be under a heel of the user's foot;
   a first subset of the one or more force sensors is positioned on the flexible substrate to be under a ball of the user's foot; and
   a first subset of the one or more force sensors is positioned on the flexible substrate to be under a toe of the user's foot.
K. The system of any one of example implementations A-J, further comprising at least one inertial measurement unit (IMU) positioned on the flexible substrate.
L. The system of example implementation K, the controller configured to:
   receive at least one of angular rate data or specific force data from the at least one IMU; and
   calculate the CoM for the user based further on the at least one of angular rate data or specific force data.
M. The system of any one of example implementations A-L, further comprising at least one additional sensor positioned on the flexible substrate for measuring one or more biometric characteristics of the user.
N. The system of example implementation M, wherein the one or more biometric characteristics of the user include at least a heart rate and an oxygen saturation of the user.
O. The system of example implementation M, wherein the at least one additional sensor is positioned on the flexible substrate to be under a lateral portion of the user's foot.
P. The system of any one of example implementations A-O, further comprising at least one of a temperature sensor or a moisture sensor positioned on the flexible substrate.
Q. The system of example implementation P, wherein the temperature sensor is configured to measure a temperature of an interior of the shoe and the moisture sensor is configured to measure a humidity level in the shoe.
R. The system of example implementation P, wherein the moisture sensor is positioned on the flexible substrate to be under a toe of the user's foot.
S. The system of any one of example implementations A-R, further comprising an antenna positioned on the flexible substrate for detecting a gait pattern of the user, wherein the gait pattern of the user is detected by measuring a resonant frequency of the user's foot during a gait cycle using the antenna.
T. The system of example implementation S, wherein the antenna is one of a coupled antenna, a meandered antenna, or a spiral antenna.
U. A method for detecting motion and balance abnormalities or inefficiencies, the method comprising:
   receiving force data from the sensor array, the sensor array comprising a plurality of force sensors positioned on a flexible substrate positioned in a shoe of a user;
   determining biomechanical characteristics for the user based on the force data, the biomechanical characteristics including at least one of a center-of-pressure (CoP), a center-of-mass (CoM), or lower extremity angles for the user;
   identifying one of a motion or balance abnormality or inefficiency for the user based on the biomechanical characteristics; and
   providing an indication of the identified abnormality or inefficiency to the user.
V. The method of example implementation U, wherein providing the indication to the user comprises causing a user device associated with the user to display a graphical user interface (GUI) that visually indicates the identified abnormality or inefficiency.
W. The method of example implementation U or V, wherein the indication further includes a recommendation for correcting the identified abnormality or inefficiency.
X. The method of any one of example implementations U-W, wherein the lower extremity angles include a predicted angle for each of an ankle, a knee, a hip, and a lower back of the user.
Y. The method of any one of example implementations U-X, wherein determining the biomechanical characteristics for the user comprises predicting the lower extremity angles using a machine learning model.
Z. The method of any one of example implementations U-Z, wherein the force data is provided as an input to the machine learning model, the machine learning model configured to predict an angle for each of an ankle, a knee, a hip, and a lower back of the user.
AA. The method of any one of example implementations Y or Z, wherein the machine learning model is a Gaussian Process Regression (GPR) model.
BB. The method of any one of example implementations U-AA, wherein the one or more force sensors comprise at least one of force sensing resistors (FSRs), piezoresistive (PZR) sensors, capacitive force sensors, or fabric-based strain sensors.
CC. The method of any one of example implementations U-BB, wherein the flexible substrate is an insole of the shoe or wherein the flexible substrate is configured to be positioned under an insole of the shoe.
DD. The method of example implementation CC, wherein the one or more force sensors comprise at least three sensors, including:
   a first subset of the one or more force sensors positioned on the flexible substrate to be under a heel of the user's foot;
   a first subset of the one or more force sensors positioned on the flexible substrate to be under a ball of the user's foot; and
   a first subset of the one or more force sensors positioned on the flexible substrate to be under a toe of the user's foot.
EE. The method of any one of example implementations U-DD, wherein the sensor array further includes at least one inertial measurement unit (IMU) positioned on the flexible substrate, the method further comprising receiving IMU data from the sensor array.
FF. The method of example implementation EE, wherein the IMU data is at least one of angular rate data or specific force data, the method further comprising calculating the CoM for the user based further on the at least one of angular rate data or specific force data.
GG. The method of any one of example implementations U-FF, wherein the sensor array further includes at least one additional sensor positioned on the flexible substrate for measuring one or more biometric characteristics of the user.
HH. The method of example implementation GG, further comprising detecting at least a heart rate and an oxygen saturation of the user based on the one or more biometric characteristics.
II. The method of example implementation GG, wherein the at least one additional sensor is positioned on the flexible substrate to be under a lateral portion of the user's foot.
JJ. The method of any one of example implementations U-II, wherein the sensor array further includes at least one of a temperature sensor or a moisture sensor positioned on the flexible substrate.
KK. The method of example implementation JJ, further comprising measuring at least one of a temperature of an interior of the shoe or a humidity level in the shoe using the at least one of a temperature sensor or a moisture sensor.
LL. The method of example implementation JJ, wherein the moisture sensor is positioned on the flexible substrate to be under a toe of the user's foot.
MM. The method of any one of example implementations U-LL, wherein the sensor array further includes an antenna positioned on the flexible substrate for detecting a gait pattern of the user, wherein the gait pattern of the user is detected by measuring a resonant frequency of the user's foot during a gait cycle using the antenna.
NN. The method of example implementation NN, wherein the antenna is one of a coupled antenna, a meandered antenna, or a spiral antenna.
OO. A system for detecting human motion and body balance, the system comprising:
   a flexible substrate configured to be positioned in a shoe of a user;
   a sensor array comprising one or more force sensors positioned on the flexible substrate; and
   a controller communicably coupled to the sensor array, the controller configured to:
      receive force data from the sensor array;
      predict an angle for each of one or more lower extremities of the user by applying the force data to a machine learning model; and
      detect an abnormality or an inefficiency in the user's movements based on the predicted angle for each of the one or more lower extremities.
PP. The system of example implementation OO, the controller further configured provide, via a user interface and responsive to detecting an abnormality or an inefficiency in the user's movements, a recommendation for correcting the abnormality or the inefficiency.
QQ. The system of example implementation OO or PP, wherein predicting an angle for each of one or more lower extremities of the user comprises:
   predicting an angle of an ankle of the user based on the force data; and
   predicting an angle of a knee of the user based on the force data and the predicted angle of the ankle.
RR. The system of example implementation QQ, wherein predicting an angle for each of one or more lower extremities of the user further comprises predicting an angle of a hip of the user based on the force data, the predicted angle of the knee, and the predicted angle of the ankle.
SS. The system of example implementation QQ, wherein predicting an angle for each of one or more lower extremities of the user further comprises predicting an angle of the L5-S1 joint of the user based on the force data, the predicted angle of the hip, the predicted angle of the knee, and the predicted angle of the ankle.
TT. The system of any one of example implementations OO-SS, wherein the machine learning model is a Gaussian Process Regression (GPR) model.
UU. The system of any one of example implementations OO-TT, the one or more force sensors comprising force sensing resistors (FSRs), piezoresistive (PZR) sensors, capacitive force sensors, or fabric-based strain sensors.
VV. The system of any one of example implementations OO-UU, wherein the flexible substrate is an insole of the shoe or wherein the flexible substrate is configured to be positioned under an insole of the shoe.
WW. The system of example implementation VV, the one or more force sensors comprising at least three sensors, wherein:
   a first subset of the one or more force sensors is positioned on the flexible substrate to be under a heel of the user's foot;
   a first subset of the one or more force sensors is positioned on the flexible substrate to be under a ball of the user's foot; and
   a first subset of the one or more force sensors is positioned on the flexible substrate to be under a toe of the user's foot.
XX. The system of any one of example implementations OO-WW, further comprising at least one additional sensor positioned on the flexible substrate for measuring one or more biometric characteristics of the user.
YY. The system of example implementation XX, wherein the one or more biometric characteristics of the user include at least a heart rate and an oxygen saturation of the user.
ZZ. The system of example implementation XX, wherein the at least one additional sensor is positioned on the flexible substrate to be under a lateral portion of the user's foot.
AAA. The system of any one of example implementations OO-ZZ, further comprising at least one of a temperature sensor or a moisture sensor positioned on the flexible substrate.
BBB. The system of example implementation AAA, wherein the temperature sensor is configured to measure a temperature of an interior of the shoe and the moisture sensor is configured to measure a humidity level in the shoe.
CCC. The system of example implementation AAA, wherein the moisture sensor is positioned on the flexible substrate to be under a toe of the user's foot.
DDD. The system of any one of example implementations ZZ-CCC, further comprising an antenna positioned on the flexible substrate for detecting a gait pattern of the user, wherein the gait pattern of the user is detected by measuring a resonant frequency of the user's foot during a gait cycle using the antenna, and wherein the gait pattern of the user is further utilized to detect an abnormality or an inefficiency in the user's movements.
EEE. The system of example implementation DDD, wherein the antenna is one of a coupled antenna, a meandered antenna, or a spiral antenna.
FFF. A method for predicting an angle of one or more lower extremities of a user, the method comprising:
   receiving force data from the sensor array, the sensor array comprising a plurality of force sensors positioned on a flexible substrate positioned in a shoe of a user;
   predicting an angle of an ankle of the user by applying the force data to a machine learning model;
   predicting an angle of a knee of the user by on the force data and the predicted angle of the ankle to the machine learning model; and
   presenting a user interface that indicates the predicted angle of the ankle and the predicted angle of the knee.
GGG. The method of example implementation FFF, wherein predicting an angle for each of one or more lower extremities of the user further comprises predicting an angle of a hip of the user based on the force data, the predicted angle of the knee, and the predicted angle of the ankle.
HHH. The method of example implementation FFF, wherein predicting an angle for each of one or more lower extremities of the user further comprises predicting an angle of the L5-S1 joint of the user based on the force data, the predicted angle of the hip, the predicted angle of the knee, and the predicted angle of the ankle.
III. The method of any one of example implementations FFF-HHH, further comprising analyzing the predicted angle of the ankle and the predicted angle of the knee to detect an abnormality in the user's movements.
JJJ. The method of example implementation III, wherein the user interface further includes a recommendation for correcting the abnormality.

KKK. The method of any one of example implementations FFF-JJJ, wherein the machine learning model is a Gaussian Process Regression (GPR) model.
LLL. The method of any one of example implementations FFF-KKK, the one or more force sensors comprising force sensing resistors (FSRs), piezoresistive (PZR) sensors, capacitive force sensors, or fabric-based strain sensors.
MMM. The method of any one of example implementations FFF-LLL, wherein the flexible substrate is an insole of the shoe or wherein the flexible substrate is configured to be positioned under an insole of the shoe.
NNN. The method of any one of example implementations FFF-MMM, the one or more force sensors comprising at least three sensors, wherein:
   a first subset of the one or more force sensors is positioned on the flexible substrate to be under a heel of the user's foot;
   a first subset of the one or more force sensors is positioned on the flexible substrate to be under a ball of the user's foot; and
   a first subset of the one or more force sensors is positioned on the flexible substrate to be under a toe of the user's foot.
OOO. The method of any one of example implementations FFF-NNN, wherein at least one additional sensor is positioned on the flexible substrate for measuring one or more biometric characteristics of the user.
PPP. The method of example implementation OOO, wherein the one or more biometric characteristics of the user include at least a heart rate and an oxygen saturation of the user.
QQQ. The method of example implementation PPP, wherein the at least one additional sensor is positioned on the flexible substrate to be under a lateral portion of the user's foot.
RRR. The method of any one of example implementations FFF-QQQ, wherein at least one of a temperature sensor or a moisture sensor is positioned on the flexible substrate.
SSS. The method of example implementation RRR, wherein the temperature sensor is configured to measure a temperature of an interior of the shoe and the moisture sensor is configured to measure a humidity level in the shoe.
TTT. The method of example implementation SSS, wherein the moisture sensor is positioned on the flexible substrate to be under a toe of the user's foot.
UUU. The method of any one of example implementations FFF-TTT, wherein an antenna is positioned on the flexible substrate for detecting a gait pattern of the user, wherein the gait pattern of the user is detected by measuring a resonant frequency of the user's foot during a gait cycle using the antenna, and wherein the gait pattern of the user is further utilized to detect an abnormality or an inefficiency in the user's movements.
VVV. The method of example implementation UUU, wherein the antenna is one of a coupled antenna, a meandered antenna, or a spiral antenna.
WWW. A system comprising:
   a flexible substrate configured to be positioned in a shoe of a user;
   a sensor array comprising:
      one or more force sensors positioned on the flexible substrate; and
      at least one of an inertial measurement unit (IMU), a temperature sensor, a moisture sensor, a gait antenna, a heart rate sensor, or an oxygen saturation sensor positioned on the flexible substrate; and
   a controller communicably coupled to the sensor array, the controller configured to:
      receive data from the sensor array, the data including force measurements from each of the one or more force sensors and at least one of an IMU measurement, a temperature measurement, a moisture measurement, a resonant frequency measurement, a heart rate measurement, or an oxygen saturation measurement for the user;
      determine biomechanical characteristics and at least one of biometric or environmental characteristics for the user based on the data; and
      provide an indication of the biomechanical characteristics and the at least one of biometric or environmental characteristics to the user.
XXX. The system of example implementation WWW, wherein providing the indication comprises transmitting, by the controller, second data including the biomechanical characteristics and the at least one of biometric or environmental characteristics to a user device associated with the user to cause the user device to display a graphical user interface (GUI).
YYY. The system of example implementation WWW or XXX, the controller further configured to detect an abnormality in the biomechanical characteristics for the user.
ZZZ. The system of example implementation YYY, wherein the indication further includes a recommendation for correcting the abnormality.
AAAA. The system of any one of example implementations WWW-ZZZ, wherein determining the biomechanical characteristics comprises predicting an angle for each of an ankle, a knee, a hip, and a lower back of the user.
BBBB. The system of any one of example implementations WWW-AAAA, wherein determining the biomechanical characteristics for the user comprises calculating the lower extremity angles using a machine learning model.
CCCC. The system of example implementation BBBB, wherein the force measurements are provided as an input to the machine learning model, the machine learning model configured to predict an angle for each of an ankle, a knee, a hip, and a lower back of the user.
DDDD. The system of any one of example implementations WWW-CCCC, wherein the one or more force sensors comprising force sensing resistors (FSRs), piezoresistive (PZR) sensors, capacitive force sensors, or fabric-based strain sensors.
EEEE. The system of any one of example implementations WWW-DDDD, wherein the flexible substrate is an insole of the shoe or wherein the flexible substrate is configured to be positioned under an insole of the shoe.
FFFF. The system of example implementation EEEE, the one or more force sensors comprising at least three sensors, wherein:
   a first subset of the one or more force sensors is positioned on the flexible substrate to be under a heel of the user's foot;
   a first subset of the one or more force sensors is positioned on the flexible substrate to be under a ball of the user's foot; and
   a first subset of the one or more force sensors is positioned on the flexible substrate to be under a toe of the user's foot.
GGGG. The system of any of example implementations WWW-FFFF, wherein the IMU measurements include at least one of angular rate data or specific force data, the controller configured to calculate a center-of-mass (CoM) for the user based further on the at least one of angular rate data or specific force data.
HHHH. The system of any of example implementations WWW-GGGG, wherein the one or more biometric characteristics of the user include at least a heart rate and an oxygen saturation of the user.
IIII. The system of any of example implementations WWW-HHHH, wherein the heart rate sensor and the oxygen saturation sensor are positioned on the flexible substrate to be under a lateral portion of the user's foot.
JJJJ. The system of any of example implementations WWW-IIII, wherein the moisture sensor is positioned on the flexible substrate to be under a toe of the user's foot.
KKKK. The system of any of example implementations WWW-JJJJ, wherein the gait antenna is configured to detect the gait pattern of the user by measuring a resonant frequency of the user's foot during a gait cycle using the antenna.
LLLL. The system of any of example implementations WWW-KKKK, wherein the gait antenna is one of a coupled antenna, a meandered antenna, or a spiral antenna.

Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

What is claimed is:

1. A system for detecting human motion and body balance, the system comprising:
a flexible substrate configured to be positioned in a shoe of a user;
a sensor array comprising one or more force sensors positioned on the flexible substrate; and
a controller communicably coupled to the sensor array, the controller configured to:
receive force data from the sensor array;
determine biomechanical characteristics for the user based on the force data, the biomechanical characteristics including at least one of a center-of-pressure (CoP), a center-of-mass (CoM), or lower extremity angles for the user; and
provide an indication of the biomechanical characteristics to the user.

2. The system of claim 1, wherein providing the indication of the biomechanical characteristics to the user comprises transmitting, by the controller, data including the biomechanical characteristics to a user device associated with the user to cause the user device to display a graphical user interface (GUI).

3. The system of claim 1, the controller further configured to detect an abnormality in the biomechanical characteristics for the user.

4. The system of claim 3, wherein the indication further includes a recommendation for correcting the abnormality.

5. The system of claim 1, wherein the lower extremity angles include a predicted angle for each of an ankle, a knee, a hip, and a lower back of the user.

6. The system of claim 1, wherein determining the biomechanical characteristics for the user comprises calculating the lower extremity angles using a machine learning model.

7. The system of claim 1, wherein the force data is provided as an input to the machine learning model, the machine learning model configured to predict an angle for each of an ankle, a knee, a hip, and a lower back of the user.

8. The system of claim 1, the one or more force sensors comprising force sensing resistors (FSRs), piezoresistive (PZR) sensors, capacitive force sensors, or fabric-based strain sensors.

9. The system of claim 1, wherein the flexible substrate is an insole of the shoe or wherein the flexible substrate is configured to be positioned under an insole of the shoe.

10. The system of claim 9, the one or more force sensors comprising at least three sensors, wherein:
a first subset of the one or more force sensors is positioned on the flexible substrate to be under a heel of the user's foot;
a first subset of the one or more force sensors is positioned on the flexible substrate to be under a ball of the user's foot; and
a first subset of the one or more force sensors is positioned on the flexible substrate to be under a toe of the user's foot.

11. The system of claim 1, further comprising at least one inertial measurement unit (IMU) positioned on the flexible substrate.

12. The system of claim 11, the controller configured to:
receive at least one of angular rate data or specific force data from the at least one IMU; and
calculate the CoM for the user based further on the at least one of angular rate data or specific force data.

13. The system of claim 1, further comprising at least one additional sensor positioned on the flexible substrate for measuring one or more biometric characteristics of the user.

14. The system of claim 13, wherein the one or more biometric characteristics of the user include at least a heart rate and an oxygen saturation of the user.

15. The system of claim 13, wherein the at least one additional sensor is positioned on the flexible substrate to be under a lateral portion of the user's foot.

16. The system of claim 1, further comprising at least one of a temperature sensor or a moisture sensor positioned on the flexible substrate.

17. The system of claim 16, wherein the temperature sensor is configured to measure a temperature of an interior of the shoe and the moisture sensor is configured to measure a humidity level in the shoe.

18. The system of claim 16, wherein the moisture sensor is positioned on the flexible substrate to be under a toe of the user's foot.

19. The system of claim 1, further comprising an antenna positioned on the flexible substrate for detecting a gait pattern of the user, wherein the gait pattern of the user is detected by measuring a resonant frequency of the user's foot during a gait cycle using the antenna.

20. The system of claim 19, wherein the antenna is one of a coupled antenna, a meandered antenna, or a spiral antenna.
